(19) United States
(12) Patent Application Publication
(10) Pub. No.: US 2003/0083304 A1
(43) Pub. Date: May 1, 2003

(54) CCAAT/enhancer binding protein-beta (C/EBPbeta) is a molecular target for cancer treatment

(76) Inventors: Robert C. Smart, Raleigh, NC (US); Songyun Zhu, Durham, NC (US); Peter J. Johnson, Frederick, MD (US); G. Esta Sterneck, Frederick, MD (US)

Correspondence Address: MYERS BIGEL SIBLEY & SAJOVEC, PO BOX 37428, RALEIGH, NC, 27627, US

(21) Appl. No.: 10/251,049

(22) Filed: Sep. 20, 2002

Related U.S. Application Data
(60) Provisional application No. 60/327,962, filed on Oct. 9, 2001.

(51) Int. Cl. A61K 48/00; C12Q 1/68; G01N 33/574; A61K 39/395

(52) U.S. Cl. 514/44; 424/155.1; 435/6; 435/7.23

(57) ABSTRACT

A method of inhibiting C/EBPβ activity in a cell is carried out by treating the cell with a compound that disrupts the C/EBPβ signaling pathway. Methods of identifying compounds useful for carrying out such treatments are also described.

### STATEMENT OF FEDERAL SUPPORT

This invention was made with United States government support under grant number CA46637 from the National Cancer Institute of the National Institutes of Health. The United States government has certain rights to this invention.

### RELATED APPLICATIONS

This application claims the benefit of provisional application serial No. 60/327,962, filed Oct. 9, 2001, the disclosure of which is incorporated by reference herein in its entirety.

### FIELD OF THE INVENTION

The present invention relates to methods and compositions for mediating cell proliferation, especially tumorigenesis.

### BACKGROUND OF THE INVENTION

GTP-binding proteins of the Ras family function as intracellular mediators of extracellular signals to regulate cell proliferation, survival and differentiation (M. Serrano, et al., Cell 88, 593-602 (1997); Mayo, et al. (1997) Science, 278:1812-1815; Bonni, et al. (1999) Science, 286:1358-1362; Gille and Downward (1999) J. Biol. Chem. 274:22033-22040). Ras proto-oncogenes are frequently mutated in tumors, and approximately 25% of human cancers contain transforming mutations in ras. 95-100% of DMBA/TPA- and DMBA/mirex-induced papillomas contain an oncogeneic mutation in the 61^{st} codon of Ha-ras, while 100% of MNNG-initiated/TPA promoted papillomas have oncogenic mutations in the 12^{th} codon of Ha-ras. Therefore, understanding oncogenic ras signaling pathways is useful for designing therapeutic strategies to prevent the development or block the growth of many classes of tumors.

Ras has numerous effectors and its pathways are multifaceted (Cambell, et al. (1998) Oncogene, 17:1395-1413; Katz and McCormick (1997) Curr. Opin. Gen. Dev. 7:75-79; Marshall, (1996) Curr. Opin. Cell Biol. 8:197-204). Ras activation can result in the phosphorylation and activation of several transcription factors through its stimulation of various pathways, which in turn regulate the expression of genes involved in numerous cell processes. The transcription factors Ets, c-jun, c-myc and NF-κB are known to have roles in oncogenic Ras-induced cellular transformation (Johnson, et al. (1994) Mol. Cell. Biol. 16:4501-4511; Langer et al. (1992) Mol. Cell. Biol., 12:5355-5362; Finco, et al. (1997) J. Biol. Chem. 272:24113-24116; Sklar, et al. (1991)) Mol. Cell. Biol., 11:3699-3710). Ras-induced apoptosis is suppressed by its activation of pro-survival pathways involving NF-κB or Rac GTPase; if these pathways are blocked, apoptosis results.

The CCAAT/enhancer binding protein (C/EBP) family of transcription factors is composed of at least five distinct members belonging to the basic leucine zipper (bZIP) class of transcription factors: C/EBPα, C/EBPβ, C/EBPδ, C/EBPε, and Ig/EBP(C/EBPγ). These proteins contain a conserved carboxy-terminal domain consisting of a basic region that recognizes specific DNA sequence and an adjacent helical structure, the leucine zipper, that mediates subunit dimerization and a variable N-terminal transactivating region. Both homo- or heterodimers of C/EBP isoforms can form and bind to C/EBP sites within the promoters/enhancers of certain genes. The expression of C/EBP isoforms is most prominent in adipocytes, hepatocytes, intestinal tissues, lung, monocytes/macrophage, ovarian follicles and epidermis.

C/EBPβ (also known as NF-IL6, IL-6, DBP, LAP, CRP2, and NF-M) is expressed in a variety of cell types (Williams, et al. (1991) Genes Dev, 5:1553-1567; Lekstrom-Himes and Xanthopoulos (1998) J. Biol. Chem., 273:28545-28548). These cell types include human and mouse keratinocytes, where C/EBPβ regulates the early events of stratified squamous differentiation. (Zhu, et al. (1999) Mol Cell Biol, 19:7181-7190); Maytin, and Habener, J. Invest. Dermatol. 110, 238-246 (1998); Oh and Smart, J. Invest. Dermatol., 110:939-945 (1998)). C/EBPβ is involved in the regulation of the expression of a number of cytokine genes, and C/EBPβ binding motifs are found in the regulatory regions of IL-1β, IL-6, IL-8, TNFα, and G-CSF.

C/EBPβ is also involved in regulating differentiation of specific mesenchymal, epithelial and hematopoietic cell types. Z. Cao, et al., Genes Dev. 5, 1538-1552 (1991); S. Natsuka, et al., Blood 79, 460-466 (1992). L. M. Scott, et al., Blood 80, 1725-1735 (1992); T. N. Seagroves, et al., Genes Dev. 12, 1917-1928 (1998); G. W. Robinson, et al., Genes Dev. 12, 1907-1916 (1998). For example, C/EBPβ plays a role in the early stages of preadipocyte differentiation and differentiation of certain cells of the myeloid lineage.

C/EBPβ-deficient mice display immune defects including lymphoproliferative disorder, distorted humoral, innate and cellular immunity, and imbalanced T-helper cell response and impaired tumor cytotoxicity and bactericidal activity of macrophages. Female mice lacking C/EBPβ are infertile due to the failure of ovarian granulosa cells to differentiate into luteal cells; these mice also demonstrate defects in the proliferation and differentiation of mammary epithelial cells.

C/EBPβ is present in cells that give rise to human and rodent tumors containing mutant ras. J. L. Bos, Cancer Res. 49, 4682-4689 (1989); A. Balmain and K. Brown, Adv. Cancer Res. 51, 147-181 (1988). C/EBPβ activity can be activated/depressed by phosphorylation through pathways stimulated by oncogenic Ras in fibroblasts, erythoblasts and P19 embryonic carcinoma cells. T. Nakajima, et al., Proc. Natl. Acad. Sci. USA 90, 2207-2211 (1993); E. Kowenz-Leutz, et al., Genes Dev. 8, 2781-2791 (1994). However, it has heretofore not been determined if C/EBPβ has a role in tumorigenesis.

### SUMMARY OF THE INVENTION

In view of the foregoing, a first aspect of the invention is a method of inhibiting C/EBPβ activity in a cell by treating the cell with (or administering to the cell) a compound that disrupts the C/EBPβ signaling pathway. Preferably, the cell is a cell that has been transformed by oncogenic ras, and even more preferably is a cancer cell.

A second aspect of the invention is a method of treating a disorder of cell proliferation in a subject in need of such treatment, by administering to the subject a compound that disrupts the C/EBPβ signaling pathway. In a preferred embodiment, the disorder is cancer.

Additional aspects of the invention include methods of screening for a compound that disrupts the C/EBPβ pathway by determining if the compound alters the level of expression of a component of the C/EBPβ pathway. In a preferred embodiment, the component of the C/EBPβ pathway is C/EBPβ. Pharmaceutical formulations that comprise such compounds are useful in the treatment of disorders of cell proliferation and are also an aspect of the invention.

The foregoing and other objects and aspects of the present invention are explained in detail in the specification and drawings set forth below.

### BRIEF DESCRIPTION OF THE DRAWINGS

FIG. 1 illustrates that C/EBPβ-null mice are completely refractory to skin tumorigenesis. C/EBPβ null ◯, wild type □ or heterozygous ⋄ mice littermates (7-8 weeks old) were treated with: (Panel A/B) a single application of 200 nmol DMBA followed one week later with thrice weekly treatment with 5 nmol TPA (n=20 C/EBPβ+/+, 21 C/EBPβ−/−, 12 C/EBPβ+/−); (Panel C/D) a single application of 2.5 μmol MNNG followed one week later with thrice weekly application of 5 nmol TPA Mice (n=22 C/EBPβ+/+, 19 C/EBPβ−/−); and (Panel E/F) 100 nmol DMBA once a week for 25 weeks (n=16 C/EBPβ+/+, 18 C/EBPβ−/−).

FIG. 2 illustrates that five C/EBPβ deficient v-Ha-ras transgenic mice display decreased tumor multiplicity and tumor size. v-Ha-ras+/−C/EBPβ+/+mice (n=16) and v-Ha-ras+/−C/EBPβ−/−mice (n=14) were treated twice weekly with 5 nmol TPA in 200 ul of acetone. (Panel A) Tumor multiplicity in v-Ha-ras+/−C/EBPβ−/−mice ◯ is decreased compared v-Ha-ras+/−C/EBPβ+/+mice □ (p<0.05, F-test). (Panel B) Tumor size distribution in v-Ha-ras+/−C/EBPβ+/+mice ▪ and v-Ha-ras+/−C/EBPβ−/−mice □ (p<0.05, Fisher's Exact Test).

FIG. 3 illustrates that oncogenic Ha-ras stimulates C/EBPβ transactivation activity. (Panels A and B): BALB/MK2 keratinocytes were transfected with pcDNA3-C/EBPβ and/or pcDNA3-Hras (12V) and the specified C/EBPdependent promoter/reporter vector. Luciferase activity was assayed forty-eight hours after transfection and expressed as fluorescent units/μg protein. Each value represents the mean±SD of triplicate dishes per treatment. Similar results were obtained from 2 repeat experiments. Inclusion of pSV-b-galactosidase and subsequent normalization of luciferase to b-galactosidase activity produced similar results to those normalized to protein levels. (Panel C): BALB/MK2 keratinocytes were transfected with Ha-ras (12V) and/or C/EBPβ and 48 hour later lysates were prepared. Equal amount of protein was loaded on 10% polyacrylamine Tris-Glycine gels and subjected to electrophoresis. Proteins were transferred and membranes were probed with polyclonal antibodies to C/EBPβ and ras.

FIG. 4 illustrates that activation of C/EBPβ by oncogenic Ha-ras involves a threonine 188 and requires the presence of the C/EBPβ transactivation domain. BALB/MK2 keratinocytes were transfected with 1.0 μg of the promoter/reporter MGF82-luc and 0.5 μg of one or more of the specified vectors. The experimental procedures were carried out as described in FIG. 3. Each value represents the mean±SD of triplicate dishes per treatment. Similar results were obtained from 2 repeat experiments. Inclusion of pSV-β-galactosidase and subsequent normalization of luciferase to β-galactosidase activity produced similar results to those normalized to protein levels.

FIG. 5 illustrates that endogenous C/EBPβ is a downstream mediator of oncogenic Ha-ras signaling in keratinocytes. Primary keratinocytes were isolated from C/EBPβ−/− or wild-type C/EBPβ+/+newborn littermates (2-3 days old) and cultured as previously described. Primary keratinocytes (˜100% confluence in culture) were transfected with pcDNA3-Hras (12V) (0.5 μg) and 1.0 μg of the specified C/EBP dependent promoter/reporter vector and processed as described in FIG. 3. Each value represents the mean±SD of triplicate dishes per treatment.

### DETAILED DESCRIPTION OF PREFERRED EMBODIMENTS

The present invention will now be described with reference to the accompanying figures and specification, in which preferred embodiments of the invention are illustrated. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise. All publications, patent applications, patents, and other references mentioned herein are incorporated by reference in their entirety.

Except as otherwise indicated, standard methods may be used for the production of cloned genes, expression cassettes, vectors (e.g., plasmids), proteins and protein fragments according to the present invention. Such techniques are known to those skilled in the art. See e.g., J. Sambrook et al., Molecular Cloning: A Laboratory Manual Second Edition (Cold Spring Harbor Laboratory, Cold Spring Harbor, N.Y., 1989), and F. M. Ausubel et al., Current Protocols In Molecular Biology (Green Publishing Associates, Inc. and Wiley-Interscience, New York, 1991).

The present invention relates to the C/EBPβ signal transduction pathway, and components thereof. The present inventors have discovered that disruption of the pathway can result in the inhibition of tumor growth in animals. The invention, therefore, relates to the implication of C/EBPβ in the development and maintenance of cell proliferation/activation, including the abnormal cellular proliferation involved in apoptosis, oncogenesis, the transformation process, and the development of cancer.

Certain embodiments of the present invention are based on the following characteristics of the transcription factor C/EBPβ, which characteristics are either known or are disclosed for the first time herein: (1) C/EBPβ can directly modulate the program of squamous differentiation in the epidermis and in isolated keratinocytes; (2) C/EBPβ activity can be modulated by post-translational modification via phosphorylation through pathways involving, for example, TGFα, Ras, PKC, PKA and calcium/calmodulin-dependent protein kinase (CaMKII), which result in the phosphorylation of specific Thr/Ser residues within C/EBPβ and greatly increase its transactivation function; (3) in fibroblasts and embryonic carcinoma cells, transfection of oncogenic ras results in the phosphorylation and activation of C/EBPβ; (4) C/EBPβ-nullizygous mice are completely refractory to tumor development in the mouse skin model of carcinogenesis using carcinogens that produce tumors with ras mutations; (5) in NIH-3T3 cells, C/EBPβ enhances Ras transformation while a dominant negative mutant of C/EBPβ inhibits Ras transformation; (6) oncogenic Ras potently stimulates C/EBPβ to activate a C/EBPresponsive promoter-reporter in keratinocytes through a pathway involving MEK1/2; and (7) the C/EBPβ-responsive promoter is activated by oncogenic Ras in wild-type but not C/EBPβ-null keratinocytes; (8) apoptosis is significantly elevated in keratinocytes of DMBA treated C/EBPβ-null mice (see Table 3 below).

Taken together, these and other observations indicate a novel role for C/EBPβ as a nuclear effector of oncogenic Ras signaling, apoptosis, transformation and tumorigenesis. Although not wishing to be bound by any particular theory of the invention, it appears that under normal signaling conditions, C/EBPβ regulates keratinocyte differentiation as well as survival. However, in the presence of oncogenic Ha-ras, the C/EBPβ pro-survival response may predominate over the differentiation pathway and clonal expansion occurs, ultimately resulting in tumor formation. These results implicate C/EBPβ as an important regulatory component of the ras transformation pathway and offer a new therapeutic target to prevent the development or block the growth of tumors containing oncogenic ras mutations.

In view of the foregoing, the present invention relates to compositions and methods for the prevention and treatment of cell proliferative disorders wherein the transcription factor C/EBPβ is involved. As described herein, cells expressing incompetent C/EBPβ pathways are no longer able to form tumors in animals, indicating that disruption of the C/EBPβ signaling pathway can be used to treat cancer in subjects in need of such treatment. Suitable subjects include, but are not limited to, mammalian and avian subjects; preferably, mammalian subjects; more preferably human, monkeys, pigs, cattle, dogs, horses, cats, sheep, and goats; and most preferably human subjects. The present invention is suitable for both medical and veterinary uses.

The invention disclosed herein provides a generalized strategy for treating and preventing cancers of any origin, either tumor-forming or non-tumor forming cancers. The inventive methods can be used to treat both the primary cancer and to prevent metastasis. The term “cancer” has its understood meaning in the art, for example, an uncontrolled growth of tissue that has the potential to spread to distant sites of the body (i.e., metastasize). As used herein, the term “cancer cell” is also intended to encompass those cells referred to as “pre-cancerous,” i.e., cells that contain mutated or damaged DNA or other components, which mutations or damage are likely to cause the cell to develop into a cancer cell.

Tumors or cancers, as defined herein, may be any tumor or cancer, primary or secondary. Exemplary cancers include osteosarcomas, angiosarcomas, fibrosarcomas and other sarcomas; papillomas; leukemias; sinus tumors; ovarian, uretal, bladder, prostate and other genitourinary cancers; colon, esophageal and stomach cancers and other gastrointestinal cancers; lung cancers; lymphomas; myelomas; pancreatic cancers; liver cancers; breast cancers; renal cancers; endocrine cancers; skin cancers; melanomas; angiomas; and brain or central nervous system (CNS) cancers. Preferred are methods of treating and preventing tumor-forming cancer.

The term “tumor” is also understood in the art, for example, as an abnormal mass of cells within a multicellular organism. Generally, the growth of the abnormal cells of the tumor exceeds and is uncoordinated with that of normal cells. Furthermore, the abnormal growth of tumor cells generally persists in an abnormal (i.e., excessive) manner after the cessation of stimuli that originally caused the abnormality in the growth of the cells. Tumors can be malignant or benign. Preferably, the inventive methods disclosed herein are used to prevent and treat malignant tumors.

By the terms “treating cancer” or “treatment of cancer,” it is intended that the severity of the cancer is reduced or the cancer is partially or entirely eliminated, or that tumor size is reduced or that the tumor is partially or entirely eliminated, as compared to that which would occur in the absence of treatment. Alternatively, these terms are intended to mean that metastasis of the cancer is reduced or eliminated, as compared to that which would occur in the absence of treatment. The term “treating cancer” may also mean that the rate of cell proliferation is decreased, as compared to that which would occur in the absence of treatment.

The methods and compositions of the present invention can be used in subjects who have already been diagnosed with cancer. As an alternative embodiment, the present invention can be carried out with individuals at risk for developing cancer. At-risk individuals include, but are not limited to, individuals with a family history of cancer, individuals who have previously been treated for cancer, individuals who have been exposed to carcinogens (e.g., heavy smokers), individuals exposed to medications or medical treatments associated with the development of cancer (e.g., estrogens or radiation therapy), individuals determined to have an increased likelihood of developing cancer by genetic testing, and individuals presenting any other clinical indicia suggesting that they have an increased likelihood of developing cancer. Alternatively stated, an at-risk individual is any individual who is believed to be at a higher risk than the general population for developing cancer.

By the terms “prevention of cancer” or “preventing cancer” it is intended that the inventive methods eliminate or reduce the incidence or onset of cancer, as compared to that which would occur in the absence of treatment. Alternatively stated, the present methods slow, delay, control, or decrease the likelihood or probability of cancer in the subject, as compared to that which would occur in the absence of treatment. In one such embodiment of the present invention, the methods of the present invention are carried out in a subject with a likelihood of having pre-cancerous mutations in certain cells (e.g., a heavy smoker with a high likelihood of having mutations in lung cells) in order to prevent or delay the onset of cancer.

The methods, compounds and compositions of the present invention are also useful in the treatment and prevention of non-cancer disorders of cell proliferation. These diseases include, but are not limited to, benign tumors, hyperplasias, hyperpigmentation of the skin, psoriasis, and any other disorder wherein cell proliferation is uncontrolled, and control of such proliferation is desired.

The present invention may be monitored for efficacy by determining a decrease in tumor size, a decrease in the number of cancerous or proliferative cells, a decrease in the rate of proliferation, or an increase in the rate of apoptosis of cancerous or proliferative cells) is achieved. Such indicia of effectiveness may be determined by techniques that are known to those skilled in the art.

Methods of the present invention may be carried out by administering to a subject a compound that inhibits C/EBPβ activity, i.e., “disrupts” the C/EBPβ pathway and related signal cascades. By disruption is meant interfering, blocking or otherwise stopping or hindering the C/EBPβ pathway or signal cascade. Compounds of the present invention render C/EBPβ unable, or limit its ability, to act as an effector of oncogenic ras, by decreasing or eliminating C/EBPβ activity. For example, a compound capable of disrupting the C/EBPβ signaling pathway would prevent activation of the oncogenic ras pathway. Such an inhibition of the C/EBPβ pathway in cells transformed with oncogenic ras would allow for restoration or maintenance of normal cell growth.

Compounds (also interchangeably referred to herein as “agents”) of the present invention may be used directly to modulate C/EBPβ signal transduction events which may lead to the development of cell proliferative disorders. Such agents may include, but are not limited to, small organic molecules, nucleic acids, proteins, peptides, or extracts of natural products which act to inhibit C/EBPβ activity and which, in turn, reduce or inhibit the development of the cell proliferative disorder of interest. Such agents may also disrupt the C/EBPβ pathway by interfering with the downstream signaling capability of C/EBPβ.

A MAPK site has been identified in C/EBPβ. Replacing Thr-188 with an Ala-188 within this site completely blocks Ras-induced activation of C/EBPβ. The MAPK inhibitor PD98059 partially blocks Ha-ras (12V)-induced activation of C/EBPβ in mouse keratinocytes. Thus, the MAPK site in C/EBPβ, which is conserved in mouse, rat, bovine and human, is important in Ras signaling. Accordingly, useful compounds of the present invention are MAPK inhibitors such as PD98059, or compounds that bind the MAPK site in C/EBPβ and thus block oncogenic ras-induced activation.

Known sites of C/EBPβ phosphorylation are Thr 217, Ser 276, and Thr 188. Replacing Ser/Thr phosphorylation sites with, for example, Ala generally inactivates the protein, while replacing the sites with the phosphate mimic Glu or increases C/EBPβ activity. Accordingly, methods of replacing Ser/Thr phosphorylation sites in C/EBPβ with inactivating amino acids (e.g., Ala) are useful in the present inventions. Additionally, compounds that prevent or block the phosphorylation of Ser/Thr phosphorylation sites in C/EBPβ are also an aspect of the present invention.

Other compounds for disrupting the C/EBP/β pathway are also an aspect of the present invention. Examples of such compounds are those peptide inhibitors of IL-6 set forth in U.S. Pat. No. 5,804,445 to Brasier et al., which is incorporated in its entirety.

In another embodiment, antibodies capable of interfering with the C/EBPβ signaling pathway may be administered for the treatment of cell proliferative disorders. For example, neutralizing antibodies which are capable of interfering with C/EBPβ signaling pathways may be administered using techniques described herein.

As used herein, the term “antibody” refers to intact molecules as well as fragments thereof, such as Fa, F(ab′)2, and Fc, which are capable of binding the epitopic determinant. The term “antibodies” includes, but are is limited to, polyclonal, monoclonal, chimeric, and single chain antibodies, and fragments thereof (e.g., fragments produced by a Fab expression library). Neutralizing antibodies, are especially preferred for therapeutic use.

Antibodies to components of the C/EBPβ pathway may be generated using methods that are well known in the art. For example, antibodies that bind components of the C/EBPβ pathway can be prepared using intact polypeptides or fragments containing small peptides of interest as the immunizing antigen. The polypeptide or oligopeptide used to immunize an animal can be derived from the translation of RNA or synthesized chemically and can be conjugated to a carrier protein, if desired. Commonly used carriers that are chemically coupled to peptides include bovine serum albumin and thyroglobulin, keyhole limpet hemocyanin. The coupled peptide is then used to immunize the animal (e.g., a mouse, a rat, or a rabbit).

For the production of antibodies, various hosts including goats, rabbits, rats, mice, humans, and others, may be immunized by injection with C/EBPβ or another component of the pathway, or any fragment or oligopeptide thereof which has immunogenic properties. Depending on the host species, various adjuvants may be used to increase immunological response. Such adjuvants include, but are not limited to, Freund's, mineral gels such as aluminum hydroxide, and surface active substances such as lysolecithin, pluronic polyols, polyanions, peptides, oil emulsions, keyhole limpet hemocyanin, and dinitrophenol. Among adjuvants used in humans, BCG (bacilli Calmette-Guerin) and Corynebacterium parvum are especially preferable.

Monoclonal antibodies to components of the C/EBPβ pathway may be prepared using any technique which provides for the production of antibody molecules by continuous cell lines in culture. These include, but are not limited to, the hybridoma technique, the human B-cell hybridoma technique, and the EBV-hybridoma technique. See, e.g., Kohler, G. et al. (1975) Nature, 256, 495-497; Kozbor, D. et al. (1985) J. Immunol. Methods 81, 31-42; Cote, R. J. et al. (1983) Proc. Natl. Acad. Sci. USA 80, 2026-2030; Cole, S. P. et al. (1984) Mol. Cell Biol. 62, 109-120.

Antibodies may also be produced by inducing in vivo production in the lymphocyte population or by screening immunoglobulin libraries or panels of highly specific binding reagents as disclosed in the literature. See, e.g., Orlandi, R. et al. (1989) Proc. Natl. Acad. Sci, 86, 3833-3837; Winter, G. et al. (1991) Nature 349:293-299.

The activity of C/EBPβ may also be inhibited (e.g., downregulated, decreased) by administering antisense nucleotides. The term “antisense”, as used herein, refers to any composition containing nucleotide sequences which are complementary to a specific DNA or RNA sequence. The term “antisense strand” is used in reference to a nucleic acid strand that is complementary to the “sense” strand. Antisense molecules include peptide nucleic acids and may be produced by any method including synthesis or transcription. Once introduced into a cell, the complementary nucleotides combine with natural sequences produced by the cell to form duplexes and block either transcription or translation. The designation “negative” is sometimes used in reference to the antisense strand, and “positive” is sometimes used in reference to the sense strand.

The terms “complementary” or “complementarity,” as used herein, refer to the natural binding of polynucleotides under permissive salt and temperature conditions by base-pairing. For example, the sequence “A-G-T” binds to the complementary sequence “T-C-A.” Complementarity between two single-stranded molecules may be “partial”, in which only some of the nucleic acids bind, or it may be complete when total complementarity exists between the single stranded molecules. The degree of complementarity between nucleic acid strands has significant effects on the efficiency and strength of hybridization between nucleic acid strands.

In order to inhibit C/EBPβ activity in cells, synthetic antisense oligonucleotides are prepared from the coding sequences for C/EBPβ found in cDNA clones. An antisense oligonucleotide consists of nucleic acid sequences corresponding to the reverse complements of C/EBPβ coding sequences or other sequences required to be present in C/EBPβ mRNA molecules for in vivo expression. The antisense oligonucleotides are introduced into cells, wherein they specifically bind to C/EBPβ mRNA molecules (and thus inhibit translation of C/EBPβ gene products), or to double-stranded DNA molecules to form triplexes. See U.S. Pat. No. 5,190,931 to Inouye, and Riordan and Martin, Nature 350, 442-443 (1991)).

Antisense oligonucleotides and nucleic acids that express the same may be made in accordance with conventional techniques. See, e.g., U.S. Pat. No. 5,023,243 to Tullis; U.S. Pat. No. 5,149,797 to Pederson et al. The length of the antisense oligonucleotide (i.e., the number of nucleotides therein) is not critical so long as it binds selectively to the intended location, and can be determined in accordance with routine procedures. In general, the antisense oligonucleotide will be from 8, 10 or 12 nucleotides in length up to 20, 30, or 50 nucleotides in length. Such antisense oligonucleotides may be oligonucleotides wherein at least one, or all, or the internucleotide bridging phosphate residues are modified phosphates, such as methyl phosphonates, methyl phosphonothioates, phosphoromorpholidates, phosphoropiperazidates and phosphoramidates. For example, every other one of the internucleotide bridging phosphate residues may be modified as described. In another non-limiting example, such antisense oligonucleotides are oligonucleotides wherein at least one, or all, of the nucleotides contain a 2′ loweralkyl moiety (e.g., C₁-C₄, linear or branched, saturated or unsaturated alkyl, such as methyl, ethyl, ethenyl, propyl, 1-propenyl, 2-propenyl, and isopropyl). For example, every other one of the nucleotides may be modified as described. See also P. Furdon et al., Nucleic Acids Res. 17, 9193-9204 (1989); S. Agrawal et al., Proc. Natl. Acad. Sci. USA 87, 1401-1405 (1990); C. Baker et al., Nucleic Acids Res. 18, 3537-3543 (1990); B. Sproat et al., Nucleic Acids Res. 17, 3373-3386 (1989); R. Walder and J. Walder, Proc. Natl. Acad. Sci. USA 85, 5011-5015 (1988).

Means for the delivery of oligonucleotides to cells include, but are not limited to, liposomes (see, e.g., K. Renneisen et al., J. Biol. Chem. 265, 16337-16342 (1990)) and introduction of expression constructs that direct the transcription of antisense oligoribonucleotides in vivo (see, e.g., O. Shohat, et al., Oncogene 1, 277-283 (1987)).

Another embodiment of the invention relates to identifying or screening compounds that are capable of disrupting the C/EBPβ pathway and/or decreasing, inhibiting or eliminating C/EBPβ activity. Compounds or agents that are screened for such capacity are referred to herein as “test compounds,” “candidate agents” or “candidate compounds.”

Candidate agents are obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of bacterial, fungal, plant and animal extracts are available or readily produced. Additionally, natural or synthetically produced libraries and compounds are readily modified through conventional chemical, physical and biochemical means. Known pharmacological agents may be subjected to directed or random chemical modifications, such as acylation, alkylation, esterification, amidification to produce structural analogs.

In one embodiment, the candidate agents are proteins. In another preferred embodiment, the candidate agents are naturally occurring proteins or fragments of naturally occurring proteins. Thus, for example, cellular extracts containing proteins, or random or directed digests of proteinaceous cellular extracts, may be used.

Generally, in a preferred embodiment of the methods herein, for example for binding assays, the C/EBPβ pathway component or the candidate agent is non-diffusibly bound to an insoluble support having isolated sample receiving areas (e.g. a microtiter plate, an array, etc.). The insoluble supports may be made of any composition to which the compositions can be bound, is readily separated from soluble material, and is otherwise compatible with the overall method of screening. The surface of such supports may be solid or porous and of any convenient shape. Examples of suitable insoluble supports include microtiter plates, arrays, membranes and beads. These are typically made of glass, plastic (e.g., polystyrene), polysaccharides, nylon or nitrocellulose, TEFLON®, etc. Microtiter plates and arrays are especially convenient because a large number of assays can be carried out simultaneously, using small amounts of reagents and samples. In some cases magnetic beads and the like are included. The particular manner of binding of the composition is not critical as long as it is compatible with the reagents and overall methods of the invention, maintains the activity of the composition and is nondiffusable. Preferred methods of binding include the use of antibodies (which do not sterically block important sites on the protein when the protein is bound to the support), direct binding to “sticky” or ionic supports, chemical crosslinking, the synthesis of the protein or agent on the surface, etc. Following binding of the protein or agent, excess unbound material is removed by washing. The sample receiving areas may then be blocked through incubation with bovine serum albumin (BSA), casein or other innocuous protein or other moiety. Also included in this invention are screening assays wherein solid supports are not used; examples of such are described below.

In a preferred embodiment, the C/EBPβ pathway component is bound to the support, and a candidate agent is added to the assay. Alternatively, the candidate agent is bound to the support and the C/EBPβ pathway component is added. Novel binding agents include specific antibodies, non-natural binding agents identified in screens of chemical libraries, peptide analogs, etc. Of particular interest are screening assays for agents that have a low toxicity for human cells. A wide variety of assays may be used for this purpose, including labeled in vitro protein-protein binding assays, electrophoretic mobility shift assays, immunoassays for protein binding, functional assays, and the like.

The determination of the binding of the candidate agent to the C/EBPβ pathway component proteins may be done in a number of ways. In a preferred embodiment, the candidate agent is labeled, and binding determined directly. For example, this may be done by attaching all or a portion of the C/EBPβ pathway component proteins to a solid support, adding a labeled candidate agent (for example a fluorescent label), washing off excess reagent, and determining whether the label is present on the solid support. Various blocking and washing steps may be utilized as is known in the art.

By “labeled” herein is meant that the compound is either directly or indirectly labeled with a label which provides a detectable signal, e.g. radioisotope, fluorescers, enzyme, antibodies, particles such as magnetic particles, chemiluminescers, or specific binding molecules, etc. Specific binding molecules include pairs, such as biotin and streptavidin, digoxin and antidigoxin etc. For the specific binding members, the complementary member would normally be labeled with a molecule which provides for detection, in accordance with known procedures. The label can directly or indirectly provide a detectable signal.

In some embodiments, only one of the components is labeled. For example, the proteins (or proteinaceous candidate agents) may be labeled at tyrosine positions using ¹²⁵I, or with fluorophores. Alternatively, more than one component may be labeled with different labels; using ¹²⁵I for the proteins, for example, and a fluorophor for the candidate agents.

In a preferred embodiment, the binding of the candidate agent is determined through the use of competitive binding assays. In this embodiment, the competitor is a binding moiety known to bind to the target molecule (i.e. C/EBPβ pathway components), such as an antibody, peptide, binding partner, ligand, etc. Under certain circumstances, there may be competitive binding as between the agent and the binding moiety, with the binding moiety displacing the agent. This assay can be used to determine candidate agents which interfere with binding between C/EBPβ pathway components and their biological binding partners. “Interference of binding” as used herein means that native binding of the C/EBPβ pathway components differs in the presence of the candidate agent. The binding can be eliminated or can be with a reduced affinity. Therefore, in one embodiment, interference is caused by, for example, a conformation change, rather than direct competition for the native binding site.

In one embodiment, the candidate agent is labeled. Either the candidate agent, or the competitor, or both, is added first to the protein for a time sufficient to allow binding, if present. Incubations may be performed at any temperature which facilitates optimal activity, typically between 4 and 40° C. Incubation periods are selected for optimum activity, but may also be optimized to facilitate rapid high through put screening. Typically between 0.1 and 1 hour will be sufficient. Excess reagent is generally removed or washed away. The second component is then added, and the presence or absence of the labeled component is followed, to indicate binding.

In a preferred embodiment, the competitor is added first, followed by the candidate agent. Displacement of the competitor is an indication that the candidate agent is binding to the C/EBPβ pathway proteins (i.e., pathway components) and thus is capable of binding to, and potentially modulating, the activity of the C/EBPβ pathway proteins. In this embodiment, either component can be labeled. Thus, for example, if the competitor is labeled, the presence of label in the wash solution indicates displacement by the agent. Alternatively, if the candidate agent is labeled, the presence of the label on the support indicates displacement.

In an alternative embodiment, the candidate agent is added first, with incubation and washing, followed by the competitor. The absence of binding by the competitor may indicate that the agent is bound to the C/EBPβ pathway component with a higher affinity. Thus, if the candidate agent is labeled, the presence of the label on the support, coupled with a lack of competitor binding, may indicate that the candidate agent is capable of binding to the C/EBPβ pathway component.

In a preferred embodiment, the methods comprise differential screening to identity agents that are capable of modulating the activity of the C/EBPβ pathway components. Such assays can be done with the C/EBPβ pathway components or cells comprising C/EBPβ pathway components. In one embodiment, the methods comprise combining an C/EBPβ pathway component and a competitor in a first sample. A second sample comprises a candidate agent, a C/EBPβ pathway component and a competitor. The binding of the competitor is determined for both samples, and a change, or difference in binding between the two samples indicates the presence of an agent capable of binding to the C/EBPβ pathway component and potentially modulating its activity. That is, if the binding of the competitor is different in the second sample relative to the first sample, the agent is capable of binding to the C/EBPβ pathway component.

Screening for agents that modulate the activity of a C/EBPβ pathway component may also be done. In a preferred embodiment, methods for screening for a agent capable of modulating the activity of C/EBPβ pathway components comprise the steps of adding a candidate agent to a sample of a C/EBPβ pathway component (or cells comprising a C/EBPβ pathway component) and determining an alteration in the biological activity of the C/EBPβ pathway component. “Modulating the activity of a C/EBPβ pathway component” includes an increase in activity, a decrease in activity, or a change in the type or kind of activity present. Thus, in this embodiment, the candidate agent should both bind to theC/EBPβ pathway component (although this may not be necessary), and alter its biological or biochemical activity as defined herein. The methods include both in vitro screening methods, as are generally outlined above, and in vivo screening of cells for alterations in the presence, distribution, activity or amount of C/EBPβ pathway components.

An alternative method for screening for agents capable of modulating the activity of a C/EBPβ pathway component. The methods comprise adding a candidate agent, as defined above, to a cell comprising C/EBPβ pathway components.

For example, measurements of C/EBPβ pathway component activity can be determined in a cell or cell population wherein a candidate agent is present and wherein the candidate agent is absent. In another example, the measurements of C/EBPβ pathway component activity are determined wherein the condition or environment of the cell or populations of cells differ from one another. For example, the cells may be evaluated in the presence or absence or previous or subsequent exposure of physiological signals, for example hormones, antibodies, peptides, antigens, cytokines, growth factors, action potentials, pharmacological agents including chemotherapeutics, radiation, carcinogenics, or other cells (i.e. cell-cell contacts).

Alternative embodiments of the invention include methods of screening for a compound that disrupts the C/EBPβ pathway by determining if a test compound alters the level of gene expression of a component of the C/EBPβ pathway. Such a method may include detecting the transcriptional or translational product of a recombinant nucleic acid construct (e.g., a vector, plasmid) comprising a nucleic acid that encodes a component of the C/EBPβ pathway, wherein the nucleic acid encoding a component of the C/EBPβ pathway is operatively linked with a reporter gene, and then determining whether the amount of the transcriptional or translational product of said reporter gene differs in the presence and absence of the test compound. In a preferred embodiment, the component of the C/EBPβ pathway is C/EBPβ.

Other embodiments include methods of screening for a compound that alters the expression of a component of the C/EBPβ pathway in a cell by contacting a cell with test compound. The cell will comprise a nucleic acid encoding a component of the C/EBPβ pathway that is operably linked to a reporter gene. The cell is then incubated under conditions appropriate for expression of the reporter gene and expression of the reporter gene is detected. A change in expression of the reporter gene is indicative of the ability of the compound to alter the gene expression of the component of the C/EBPβ pathway.

Using the foregoing methods, useful therapeutic compounds are identified. Useful compounds with pharmacological activity are those compounds that are able to enhance or interfere with the activity of at least one of the C/EBPβ pathway components. The compounds having the desired pharmacological activity may be administered in a pharmaceutically acceptable carrier (i.e., a pharmaceutical formulation) to a host or subject

Pharmaceutical formulations of the present invention comprise compounds with pharmacological activity (as identified using methods of the present invention) in a pharmaceutically acceptable carrier. Compounds with pharmacological activity are those compounds identified by methods of the present invention that are useful in disrupting or inhibiting the C/EBPβ pathway. Suitable pharmaceutical formulations include those suitable for inhalation, oral, rectal, topical, (including buccal, sublingual, dermal, vaginal and intraocular), parenteral (including subcutaneous, intradermal, intramuscular, intravenous and intraarticular) and transdermal administration. The compositions may conveniently be presented in unit dosage form and may be prepared by any of the methods well known in the art. The most suitable route of administration in any given case may depend upon the anatomic location of the condition being treated in the subject, the nature and severity of the condition being treated, and the particular pharmacologically active compound which is being used. The formulations may conveniently be presented in unit dosage form and may be prepared by any of the methods well known in the art.

In the manufacture of a medicament according to the invention (the “formulation”), pharmacologically active compounds or the physiologically acceptable salts thereof (the “active compounds”) are typically admixed with, inter alia, an acceptable carrier. The carrier must, of course, be acceptable in the sense of being compatible with any other ingredients in the formulation and must not be deleterious to the patient. The carrier may be a solid or a liquid, or both, and is preferably formulated with the compound as a unit-dose formulation, for example, a tablet, which may contain from 0.5% to 99% by weight of the active compound. One or more active compounds may be incorporated in the formulations of the invention, which formulations may be prepared by any of the well known techniques of pharmacy consisting essentially of admixing the components, optionally including one or more accessory therapeutic ingredients.

Formulations suitable for oral administration may be presented in discrete units, such as capsules, cachets, lozenges, or tablets, each containing a predetermined amount of the active compound; as a powder or granules; as a solution or a suspension in an aqueous or non-aqueous liquid; or as an oil-in-water or water-in-oil emulsion. Such formulations may be prepared by any suitable method of pharmacy which includes the step of bringing into association the active compound and a suitable carrier (which may contain one or more accessory ingredients as noted above). In general, the formulations of the invention are prepared by uniformly and intimately admixing the active compound with a liquid or finely divided solid carrier, or both, and then, if necessary, shaping the resulting mixture. For example, a tablet may be prepared by compressing or molding a powder or granules containing the active compound, optionally with one or more accessory ingredients. Compressed tablets may be prepared by compressing, in a suitable machine, the compound in a free-flowing form, such as a powder or granules optionally mixed with a binder, lubricant, inert diluent, and/or surface active/dispersing agent(s). Molded tablets may be made by molding, in a suitable machine, the powdered compound moistened with an inert liquid binder. Formulations for oral administration may optionally include enteric coatings known in the art to prevent degradation of the formulation in the stomach and provide release of the drug in the small intestine.

Formulations of the present invention suitable for parenteral administration comprise sterile aqueous and non-aqueous injection solutions of the active compound, which preparations are preferably isotonic with the blood of the intended recipient. These preparations may contain anti-oxidants, buffers, bacteriostats and solutes which render the formulation isotonic with the blood of the intended recipient. Aqueous and non-aqueous sterile suspensions may include suspending agents and thickening agents. The formulations may be presented in unit\dose or multi-dose containers, for example sealed ampoules and vials, and may be stored in a freeze-dried (lyophilized) condition requiring only the addition of the sterile liquid carrier, for example, saline or water-for-injection immediately prior to use. Extemporaneous injection solutions and suspensions may be prepared from sterile powders, granules and tablets of the kind previously described. For example, in one aspect of the present invention, there is provided an injectable, stable, sterile composition comprising a compound of the present invention, in a unit dosage form in a sealed container. The compound or salt is provided in the form of a lyophilizate which is capable of being reconstituted with a suitable pharmaceutically acceptable carrier to form a liquid composition suitable for injection thereof into a subject. The unit dosage form typically comprises from about 10 mg to about 10 grams of the compound or salt. When the compound or salt is substantially water-insoluble, a sufficient amount of emulsifying agent which is physiologically acceptable may be employed in sufficient quantity to emulsify the compound or salt in an aqueous carrier. One such useful emulsifying agent is phosphatidyl choline.

Further, the present invention provides liposomal formulations of the compounds disclosed herein and salts thereof. The technology for forming liposomal suspensions is well known in the art. When the compound or salt thereof is an aqueous-soluble salt, using conventional liposome technology, the same may be incorporated into lipid vesicles. In such an instance, due to the water solubility of the compound or salt, the compound or salt will be substantially entrained within the hydrophilic center or core of the liposomes. The lipid layer employed may be of any conventional composition and may either contain cholesterol or may be cholesterol-free. When the compound or salt of interest is water-insoluble, again employing conventional liposome formation technology, the salt may be substantially entrained within the hydrophobic lipid bilayer which forms the structure of the liposome. In either instance, the liposomes which are produced may be reduced in size, as through the use of standard sonication and homogenization techniques.

Of course, the liposomal formulations containing the pharmaceutically active compounds identified with the methods described herein may be lyophilized to produce a lyophilizate which may be reconstituted with a pharmaceutically acceptable carrier, such as water, to regenerate a liposomal suspension.

Agents intended to be administered intracellularly may be administered using techniques well known to those of ordinary skill in the art. For example, such agents may be encapsulated into liposomes, then administered as described above. Liposomes are spherical lipid bilayers with aqueous interiors. All molecules present in an aqueous solution at the time of liposome formation are incorporated into the aqueous interior. The liposomal contents are both protected from the external microenvironment and, because liposomes fuse with cell membranes, are efficiently delivered into the cell cytoplasm. Additionally, due to their hydrophobicity, small organic molecules may be directly administered intracellularly.

Other pharmaceutical formulations may be prepared from the water-insoluble compounds disclosed herein, or salts thereof, such as aqueous base emulsions. In such an instance, the formulation will contain a sufficient amount of pharmaceutically acceptable emulsifying agent to emulsify the desired amount of the compound or salt thereof. Particularly useful emulsifying agents include phosphatidyl cholines, and lecithin.

In addition to the pharmacologically active compounds, the pharmaceutical formulations may contain other additives, such as pH-adjusting additives. In particular, useful pH-adjusting agents include acids, such as hydrochloric acid, bases or buffers, such as sodium lactate, sodium acetate, sodium phosphate, sodium citrate, sodium borate, or sodium gluconate. Further, the compositions may contain microbial preservatives. Useful microbial preservatives include methylparaben, propylparaben, and benzyl alcohol. The microbial preservative is typically employed when the formulation is placed in a vial designed for multidose use. Of course, as indicated, the pharmaceutical formulations of the present invention may be lyophilized using techniques well known in the art.

The therapeutically effective dosage of any specific pharmacologically active compound identified by methods of the invention, the use of which compounds is in the scope of present invention, will vary somewhat from compound to compound, and subject to subject, and will depend upon the condition of the patient and the route of delivery.

The examples, which follow, are set forth to illustrate the present invention, and are not to be construed as limiting thereof.

### EXAMPLE 1

### Resistance of C/EBPβ Nullizygous Mice to DMBA/TPA-Induced Carcinogenesis

C/EBPβ-nullizygous mice in the mouse skin multistage model of carcinogenesis were used to determine if C/EBPβ is involved in ras tumorigenesis. The mouse skin model is one of the best-defined in vivo paradigms of experimental epithelial carcinogenesis. Furthermore, there is evidence that the mutational-activation of ras plays a central role in skin tumor development induced by a variety of carcinogens. Yuspa, Cancer Res, 54:1178-89 (1994); Frame, et al., Philos. Trans. R. Soc. Lond. B. Biol. Sci., 353:839-845) (1998).

For one series of experiments, C/EBPβ deficient mice were generated by homologous recombination. E. Sterneck, et al., Genes Dev. 11, 2153-2162 (1997). The mutant and wild type mice were generated by mating heterozygous 129/Sv females to heterozygous males from the 6^{th}-8^{th} generation backcross into the C57BL/6 strain. Initiation with a single dose of the carcinogen, 7,12-dimethylbenz[a]anthracene (DMBA), followed by repetitive treatment with the tumor promoter, TPA, results in the appearance of squamous papillomas, 95-100% of which contain an A->T¹⁸² mutation in Ha-ras. See A. Balmain, K. Brown, Adv. Cancer Res. 51, 147-181 (1988); M. Quintanilla, et al., Nature 322, 78-80 (1986); G. J. Moser, et al., Carcinogenesis 14, 1153-1160 (1993).

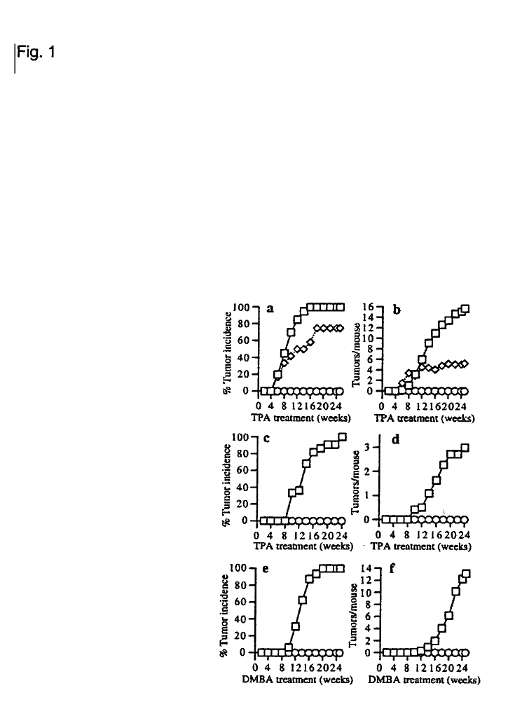

C/EBPβ nullizygous and wild-type littermates were initiated with 200 nmol DMBA. One week later these mice were promoted thrice weekly with 5 nmol TPA for 25 weeks. Wild-type mice developed an average of 15 squamous papillomas/mouse and exhibited a 100% incidence of papillomas (FIG. 1, panels A and B). In contrast, C/EBPβ nullizygous mice were completely refractory to papilloma development and no papillomas appeared after 25 weeks of promotion. In some groups of mutant mice TPA promotion was continued for 35 weeks, but no tumors developed within this time (data not shown). C/EBPβ heterozygous mice express a level of C/EBPβ protein in keratinocytes that is intermediate between that of wild-type and C/EBPβ-deficient animals. See H. -S. Oh, R. C. Smart, J. Invest. Dermatol. 110, 939-945 (1998). Accordingly, C/EBPβ heterozygous mice were partially resistant to DMBA/TPA-induced carcinogenesis (FIG. 1 panels A and B), indicating that the tumor modifying effect of C/EBPβ is gene dosage dependent. No significant sex difference in tumor response was observed.

C/EBPβ can be phosphorylated via a PKC pathway, and in hepatocytes this event is required for TPA-induced mitogenesis. See C. Trautwein, et al., J. Clin. Invest 93, 2554-2561 (1994); M. Buck, et al., Molecular Cell 4, 1087-1092 (1999). In view of these observations, experiments were carried out to determine whether TPA-induced keratinocyte proliferation was altered in epidermis of C/EBPβ-null mice. Wild-type and C/EBPβ-null mice were treated with a single application of 5 nmol TPA/200 μl acetone or acetone alone or they were treated thrice weekly for 1 month with 5 nmol TPA/200 μl acetone or acetone alone. BrdU labeling was conducted by a single dose i.p. injection of BrdU (100 mg/kg body weight) 18 hours after the last TPA treatment, one hour later the animals were euthanized and immunochemical staining of BrdU positive cells was performed as described. See H. -S. Oh, R. C. Smart, J. Invest. Dermatol. 110, 939-945 (1998); S. Zhu, et al., Mol. Cell. Biol. 19, 7181-7190 (1999).

Data are expressed as the mean±SD from at least 3 different mice. Each value for wild type mice and similarly treated C/EBP null mice within each category was not significantly different p>0.05 as determined by the student t-test.

No significant differences were observed between wild-type and C/EBPβ nullizygous mice after single or multiple treatment with TPA (Table 1). Immunohistochemical staining for keratinocyte differentiation markers, keratin 1, keratin 10, involucrin and loricrin in the epidermis of TPA-treated mice did not reveal any major differences between the two genotypes (data not shown).

TABLE 1
Effect of TPA treatment on epidermal cell proliferation
in wild-type and C/EBPβ-null mice.

| | |
|---|---|
| Nucleated Cell Layers | BrdU Positive Cells (%) |

Single Treatment

| | | |
|---|---|---|
| Acetone | | |
| Wild-type | 1.3 ± 0.1 | 4.6 ± 1.3 |
| C/EBPβ −/− | 1.5 ± 0.1 | 7.4 ± 3.7 |
| TPA | | |
| Wild-type | 1.8 ± 0.3 | 39.7 ± 8.5 |
| C/EBPβ −/− | 2.0 ± 0.9 | 43.9 ± 3.7 |

Multiple Treatment

| | | |
|---|---|---|
| Acetone | | |
| Wild-Type | 1.3 ± 0.1 | 6.0 ± 1.2 |
| C/EBPβ −/− | 1.7 ± 0.2 | 10.9 ± 5.6 |
| TPA | | |
| Wild-type | 3.8 ± 1.6 | 32.2 ± 7.7 |
| C/EBPβ −/− | 3.7 ± 0.6 | 30.6 ± 8.7 |

C/EBPβ has been implicated in the regulation of COX2 and TNFα expression, and both TNFα null and COX2 null mice are partially resistant to DMBA/TPA-induced carcinogenesis. See S. T. Reddy, et al., J. Biol. Chem. 275, 3107-3113 (2000); C. Drouet, et al., J. Immunol. 147, 1694-1700 (1991); R. J. Moore, et al., Nat. Med. 5, 828-31 (1999); R. Langenbach, et al., Biochem. Pharmacol. 58, 1237-1246 (1999). However, TNFα mRNA and COX2 protein expression was not altered in untreated or TPA-treated C/EBPβ deficient mice compared to similarly treated wild type mice (data not shown). These results indicate that TNFα and COX2 expression as well as TPA-induced proliferative responses in the epidermis of C/EBPβ-null mice are normal and thus, are not responsible for the resistance of C/EBPβ-null mice to DMBA/TPA-induced tumorigenesis.

### EXAMPLE 2

### C/EBPβ Deficient Mice are Resistant to Tumorigenesis by Other Agents

Mice were exposed to the direct carcinogen N-methyl-N′-nitro-N-nitrosoguanidine (MNNG) followed by TPA promotion in order to exclude the possibilities that C/EBPβ nullizygous mice are refractory to DMBA initiation due to their inability to convert DMBA to the ultimate carcinogenic species, and/or that their resistance to tumorigenesis was unique to the DMBA/TPA protocol. The MNNG/TPA carcinogenesis protocol produces papillomas with oncogenic mutations in the 12^{th} codon of Ha-ras and Ki-ras. A. Balmain, K. Brown, Adv. Cancer Res. 51, 147-181 (1988); S. H. Yuspa, Cancer Res. 54, 1178-89 (1994); I. Rehman, et al., Mol. Carcinog. 27, 298-307 (2000). Wild-type mice displayed a 100% incidence of papillomas with approximately 3 papillomas/mouse, while C/EBPβ null littermates did not develop any tumors (FIG. 1, panels C and D).

Since both MNNG- and DMBA-initiated mice were treated with TPA, it was possible that the inability of C/EBPβ-null mice to develop tumors resulted from their inability to respond to TPA or to an initiation/promotion protocol. Accordingly, a complete carcinogenesis protocol in which wild-type and C/EBPβ-null mice were treated once weekly with DMBA was used. All of the wild-type mice developed papillomas with an average of 12 papillomas/mouse while C/EBPβ mutant mice were again completely resistant to carcinogenesis (FIG. 1, panels E and F). It was also observed that C/EBPβ null mice were refractory to DMBA-initiation followed by promotion with the non-phorbol ester tumor promoter, mirex (data not shown). Thus, C/EBPβ nullizygous mice are fully resistant to tumorigenesis induced by a variety of carcinogens, tumor promoters and carcinogenesis protocols. Since these diverse protocols and agents all result in papillomas that contain mutant oncogenic Ha-ras or Ki-ras in normal mice, these data suggest that C/EBPβ may be a downstream mediator of oncogenic Ras tumorigenesis.

### EXAMPLE 3

### Decreased Tumor Multiplicity and Tumor Size in C/EBPβ-Deficient v-Ha-ras Transgenic Mice

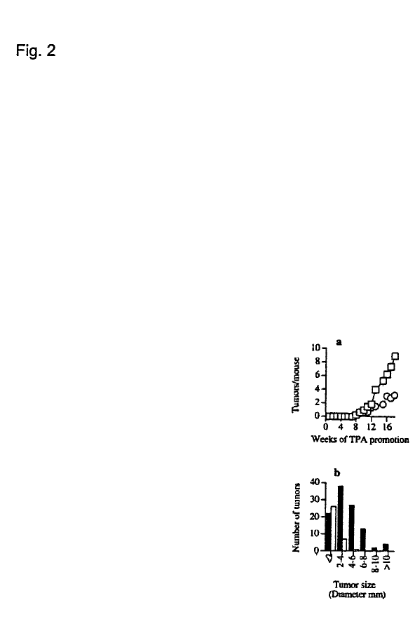

To further demonstrate a relationship between Ras and C/EBPβ in skin tumorigenesis, C/EBPβ null mice were crossed with Tg.AC transgenic mice. Tg.AC mice contain a v-Ha-ras transgene and are susceptible to skin tumor development in the absence of carcinogen exposure. See J. W. Spalding et al., Carcinogenesis 14, 1335-1341 (1993); A. Leder, et al., Proc. Natl. Acad. Sci. 87, 9178-9182 (1990). Tumorigenesis in Tg.AC mice does require a promoting stimulus such as wounding or treatment with a tumor promoter. As shown in FIG. 2, panel A, TPA-treated C/EBPβ deficient mice carrying the v-Ha-ras transgene developed ˜65% fewer skin tumors than C/EBPβ+/+mice carrying the v-Ha-ras transgene and the tumor size (FIG. 2, panel B) was reduced by greater than 60% in the C/EBPβ null mice (4.1±2.4 mm C/EBPβ+/+vs 1.7.1±1.0 mm C/EBPβ−/−p<0.01 student t-test). While there was not a complete ablation of tumor development in the C/EBPβ null mice carrying the v-Ha-ras transgene, it is clear that C/EBPβ affects the development and growth of Ras-induced papillomas. These results support a direct role for C/EBPβ as a nuclear effector of Ras-mediated tumorigenesis. These results also suggest that C/EBPβ may have an additional role in carcinogen-induced tumorigenesis.

### EXAMPLE 4

### Oncogenic Ras Stimulates C/EBPβ-Transactivation Activity

To ascertain if an oncogenic Ha-ras pathway can stimulate C/EBPβ activity in keratinocytes, BALB/MK2 were transfected with keratinocytes with C/EBPβ and/or oncogenic Ha-ras (12V) and a luciferase reporter gene fused to different lengths of the C/EBPdependent myelomonocytic growth factor (MGF) promoter. S. Zhu, et al., Mol. Cell. Biol. 19, 7181-7190 (1999) and E. Sterneck, et al., EMBO. J. 11, 115-126 (1992). pMGF-40 contains a 40 bp portion of the MGF promoter that lacks C/EBP sites, while pMGF-82 contains an additional 42 bp region of the promoter that contains two C/EBP binding sites.

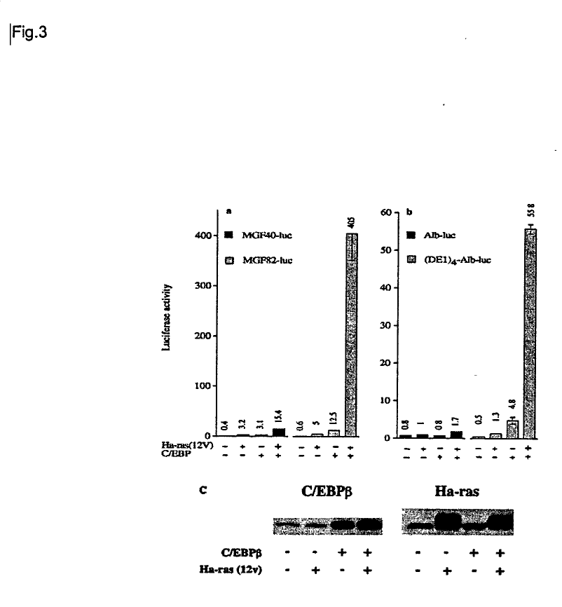

Co-transfection of oncogenic Ha-ras (12V) and C/EBPβ resulted in a 30- and 80-fold increases, respectively in pMGF-82 reporter activity over that observed with C/EBPβ or oncogenic Ha-ras (12V) alone (FIG. 3, panel A). When MGF-40 was substituted for pMGF-82, cotransfection of oncogenic Ha-ras (12V) and C/EBPβ caused only a 5-fold increase in luciferase activity, demonstrating that C/EBP binding sites are required for the synergistic interaction between Ha-ras and C/EBPβ. Similar results were obtained using a minimal albumin promoter with four tandem C/EBP sites {(DE1)₄-Alb-luc} (FIG. 3 panel B). See S. C. Williams, et al., EMBO. J. 14, 3170-3183 (1995). Western blot analysis of cell lysates from BALB/MK2 cells co-transfected with C/EBPβ and Ha-ras (12V) demonstrated that the observed synergistic effect on C/EBPresponsive promoter-reporter activity is not due to increased Ha-ras (12V) or C/EBPβ expression (FIG. 3, panel C).

### EXAMPLE 5

### Activation of C/EBPβ by Oncogenic Ha-ras Involves a Threonine 188 and Requires the Presence of the C/EBPβ Transactivation Domain

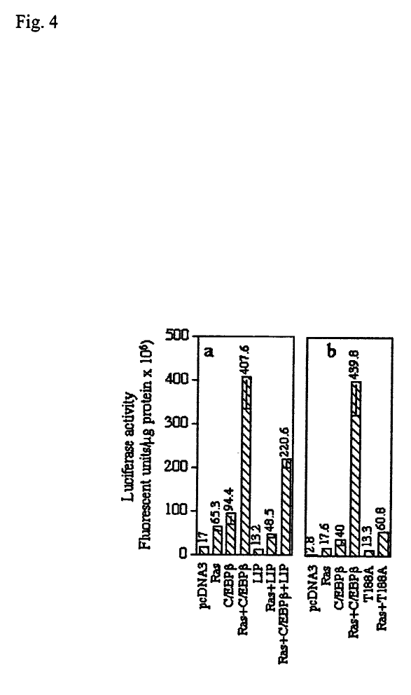

Co-transfection of Ha-ras (12V) with a truncated form of C/EBPβ (LIP; liver inhibitory protein) that lacks the N-terminal activation domain but retains the bZIP DNA-binding and leucine zipper domain (44) did not increase the activity of the pMGF-82 reporter (FIG. 4A). In fact, LIP inhibited the activation of wild-type C/EBPβ by Ha-ras (12V) by ˜50%, which is consistent with its known role as a dominant negative inhibitor of C/EBPβ (FIG. 4A). See P. Descombes, U. Schibler Cell 67 569-579 (1991) Previous studies have identified an ERK1/2 phosphorylation site (T 188) in C/EBPβ, and substituting T188 with alanine diminished Ras activation of C/EBPβ See T Nakajima et al Proc. Natl. Acad. Sci. USA 90, 2207-2211 (1993). Therefore, we tested the Ras-responsiveness of a C/EBPβ mutant containing the T188A substitution. Oncogenic Ha-ras-induced stimulation of C/EBPβ activity was abolished in this mutant. (FIG. 4B) Thus, an oncogenic Ha-ras pathway can activate C/EBPβ in keratinocytes and this activation is dependent upon threonine 188 of C/EBPβ.

### EXAMPLE 6

### Endogenous C/EBPβ Can Mediate Ras Signaling

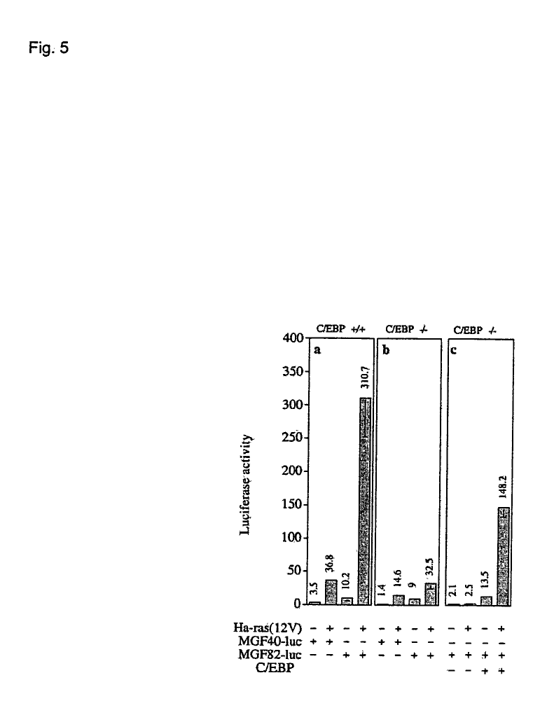

Primary keratinocytes isolated from C/EBPβ-nullizygous and wild-type mice were transfected with with oncogenic Ha-ras and the C/EBP promoter-reporter constructs. Transfection of Ha-ras (12V) into wild-type keratinocytes resulted in a 30-fold increase in pMGF-82 reporter activity while in C/EBPβ-nullizygous keratinocytes Ha-ras (12V) caused less than a 4-fold increase (FIGS. 5A and B). The Ras-induced increase in promoter activity required C/EBP binding sites (FIG. 5A). Similar results were obtained with the (DE1)₄-Alb-luc reporter (data not shown). Ectopic expression of C/EBPβ in C/EBPβ-null keratinocytes restored responsiveness to oncogenic Ras (FIG. 5B). Thus, endogenous C/EBPβ is a downstream mediator of oncogenic Ha-ras signaling in primary keratinocytes.

### EXAMPLE 7

### C/EBPβ Augments Ras-Induced Transformation of NIH-3T3 Cells

The NIH-3T3 focus assay has been widely used to identify pathways and genes that cooperate with Ras to induce transformation. To examine the role of C/EBPβ in NIH-3T3 transformation, it was first confirmed that oncogenic Ras could stimulate C/EBPβ to activate a C/EBP-responsive promoter-reporter in NIH-3T3 cells (data not shown). Next it was determined whether C/EBPβ has the capacity to transform cells and/or cooperate with oncogenic Ha-ras to increase its transforming potential in the NIH-3T3 focus formation assay. Transfection of C/EBPβ alone did not induce NIH 3T3 transformation, (Table 2) showing that this transcription factor does not possess intrinsic transforming activity. Co-transfection of C/EBPβ enhanced the transformation potential of oncogenic Ha-ras (12V), producing a ˜1.7 fold increase in the number of transformed foci compared to Ha-ras (12V) alone (Table 2). Importantly, we observed that co-transfection of LIP or C/EBPβ T188A inhibited Ha-ras (12V)-induced transformation, indicating an important role for endogenous C/EBPβ in ras-induced transformation of NIH-3T3 cells. C/EBPβ also enhanced the transforming potential of oncogenic Raf, lending further support for a Ras-Raf-ERK-C/EBPβ pathway. In contrast to C/EBPβ, neither C/EBPα or C/EBPδ enhanced the transforming activity of oncogenic Ha-ras (12V) (Table 2). Thus not all C/EBP family members are capable of augmenting Ras transformation.

TABLE 2
C/EBPβ enhances oncogenic Ha-ras-induced transformation
of NIH-3T3 cells

| | | Transformed Foci/Dish |
|---|---|---|
| 10 | ng pcDNA3 | 0.0 ± 0.0 |
| 10 | ng C/EBPβ | 0.0 ± 0.0 |
| 10 | ng Ha-ras (12V) | 35.3 ± 3.5 |
| +10 | ng C/EBPβ | 57.7 ± 1.5* |
| 10 | ng Ha-ras (12V) | 34.7 ± 3.5 |
| +10 | ng C/EBPβ | 58.9 ± 3.8* |
| +10 | ng LIP | 20.7 ± 2.4* |
| +10 | ng C/EBPβ (T188A) | 14.0 ± 2.0* |
| 10 | ng Ha-ras | 25.0 ± 1.0 |
| +10 | ng C/EBPα | 25.0 ± 4.4 |
| +10 | ng C/EBPδ | 22.3 ± 2.3 |
| 100 | ng Raf (22W) | 29.3 ± 4.5 |
| +10 | ng C/EBPβ | 47.0 ± 1.7* |

Data are expressed as transformed foci/plate and each value represents the mean±SD of triplicate dishes per treatment. All experiments were repeated at least two times and similar results were obtained in each experiment. Empty vectors or vectors containing C/EBPβ, C/EBPβ (T188A) or LIP did not produce any transformed foci at all doses examined (1 ng-1000 ng/plate). * significantly different from the value of cells transfected with Ha-ras (12V) or Raf (22W) alone as determined by the Student t-test, p<0.01.

### EXAMPLE 8

### Mapping Sites of Phosphorylation on C/EBPβ Induced by Oncogenic Ha-ras (12V)

Expression of Ha-ras (12V) stimulates the ability of C/EBPβ to transactivate a C/EBPdependent promoter by 30-fold or more in keratinocytes and in L cell fibroblasts, suggesting that C/EBPβ is a nuclear target of the ras signaling pathway. Stimulation of C/EBPβ activity by ras is likely to occur via phosphorylation on specific residues. C/EBPβ contains a site (Thr188) that is phosphorylated by MAPK in response to ras signaling. T. Nakajima et al., Proc. Natl. Acad. Sci. USA 90, 2207-2211 (1993).

However, the inventors have found a C/EBPβ mutant harboring Ala at position 188 is still stimulated by oncogenic ras, although somewhat less efficiently than wt C/EBPβ, indicating that there are additional sites of phosphorylation induced by ras signaling. These sites are mapped by metabolic ³²P labeling of C/EBPβ transiently expressed in BALB/MK keratinocytes cells in the presence or absence of Ha-ras (12V). Two days after transfection with a C/EBPβ expression vector±Ha-ras (12V) using FuGENE reagent (Boehringer Mannheim), the cells are labeled with 5-10 mCi ³²P orthophosphate for 5 hr. Whole cell lysates are prepared using RIPA buffer and the labeled C/EBPβ proteins immunoprecipitated with a peptide antibody raised against the amino terminus of C/EBPβ.

After SDS-PAGE separation and transfer to an Immobilon P membrane, the labeled proteins are visualized by autoradiography. The C/EBPβ bands are then excised and digested with sequencing grade trypsin (Boehringer Mannheim). The tryptic peptides are separated by C4 reversed phase HPLC and the fractions analyzed by scintillation counting. Ras-dependent phosphorylation events are indicated by the appearance of new peaks induced by coexpression of Ha-ras (12V) or increased levels of ³²P in peptides that contain label in the absence of ras. The positions of phosphorylated residues in tryptic peptides are determined by Edman sequencing using an ABI sequencer adapted to collect the products of each degradative cycle for ³²P quantitation.

³²P released at each cycle is measured by spotting the sample onto filter paper followed by phosphoimage analysis (Molecular Dynamics). Each peptide is also subjected to ³²P phosphoamino acid analysis to determine the identity of the residue phosphorylated in response to ras (P-Thr, P-Ser, or P-Tyr). In the event that large labeled peptides are obtained, secondary digestion with other site-specific proteases are performed and the products separated again by HPLC and subjected to phosphoamino acid analysis and sequencing. To aid in the identification of novel ras-induced sites, tryptic phosphopeptide analysis of labeled C/EBPβ mutants containing alanine substitutions in the known phosphorylation sites (Thr188, Thr217, and Ser276) is carried out.

### EXAMPLE 9

### Site-Directed Mutagenesis of Known and Newly-Identified C/EBPβ Phosphorylation Sites

Alanine substitution mutations in known and novel phosphorylation sites are introduced into the murine C/EBPβ gene (pcDNA 3.1-mC/EBPβ) by site-directed mutagenesis using the Stratagene QuickChange® mutagenesis system according to the manufacturer's recommendations. The mutations are constructed both singly and in combination, and the altered codons are verified by DNA sequencing. Mutants containing alanine substitutions in novel ras-induced sites are tested for the loss of Ha-ras (12V) stimulation in transactivation assays and for the absence of ras-dependent phosphorylation events in ³²P labeling experiments. Mutants containing phosphate mimic Glu or Asp substitutions are also constructed and tested for enhanced activity in transactivation assays in the absence of Ha-ras (12V). The Glu/Asp mutants are examined for their ability to promote oncogenic transformation of NIH 3T3 cells to determine if their transforming potential is enhanced compared to the wt C/EBPβ gene.

### EXAMPLE 10

### Apoptosis is Elevated in DMBA-Treated C/EBPβ Null Mice

The lack of tumor development in carcinogen treated C/EPBβ-null mice could be due to apoptosis of C/EBPβ-deficient keratinoyctes that have acquired oncogenic Ha-ras lesions. To examine this possibility we treated mice with DMBA and scored the number of apoptotic keratinocytes in C/EBPβ-null and wild type epidermis using the cytological parameters described in the Methods. Compared to wild type mice, C/EBPβ-null mice exhibited a 17-fold increase in the number of basal apoptotic keratinocytes (Table 3), indicating that C/EBPβ functions as a survival factor in DMBA/Ras-induced oncogenesis. Similar fold increases in apoptotic cells were observed using TUNEL staining (data not shown). To determine if C/EBPβ-null mice also display increased apoptosis in response to UVB irradiation, a potent inducer of apoptosis and DNA damage, wild type and C/EBPβ-null mice were irradiated with UVB doses of 50, 100 and 200 mJ/cm². While all UVB doses increased the number of apoptotic epidermal keratinocytes, there was no difference between wild type and mutant mice (data not shown). These results show that the enhanced apoptosis in DMBA-treated C/EBPβ-null mice is stimulus specific and that DNA damage alone is not sufficient to elicit increases in apoptosis in epidermal keratinocytes of C/EBPβ-null mice.

TABLE 3
Apoptosis is significantly elevated in epidermal keratinocytes
of DMBA-treated C/EBPβ-null mice.

Apoptotic Keratinocytes (%)

| | Wild type | C/EBPβ −/− |
|---|---|---|
| Acetone treated | 0.02 ± 0.02 | 0.04 ± 0.01 |
| DMBA treated | 0.10 ± 0.02^{a} | 1.73 ± 0.14^{ab} |

Mice (3/group) were treated with a single application of 400 nmol DMBA/200 μl acetone of acetone alone. More than 4000 basal keratinocytes were counted for each individual mouse.
^{a}significantly different from acetone-treated group (p < 0.01) as determined by student t-test
^{b}significantly different from wild type DMBA-treated group (p < 0.01) as determined by student t-test.

The foregoing examples are illustrative of the present invention, and are not to be construed as limiting thereof. The invention is described by the following claims, with equivalents of the claims to be included therein.

That which is claimed is:

1. A method of inhibiting C/EBPβ activity in a cell, comprising treating the cell with a compound that disrupts the C/EBPβ signaling pathway.

2. The method according to claim 1, wherein the cell has been transformed with oncogenic ras.

3. The method according to claim 1, wherein the compound binds the C/EBPβ protein.

4. The method according to claim 1, wherein the compound interferes with the expression of the C/EBPβ protein.

5. The method according to claim 1, wherein the compound binds a component of the C/EBPβ signaling pathway.

6. The method according to claim 1, wherein the cell is a cancerous cell.

7 The method according to claim 1, wherein the compound is administered to the cell in vivo.

8. The method according to claim 1, wherein the compound is administered to the cell in vitro.

9. The method according to claim 1, wherein the compound interferes with the post-translational phosphorylation of C/EBPβ in the cell.

10. The method according to claim 1, wherein the compound disrupts phosphorylation of the C/EBP protein at a phosphorylation site selected from the group consisting of Thr 217, Ser 276, and Thr 188.

11. The method according to claim 1, wherein the compound is a MAPK-inhibitor.

12. The method according to claim 1, wherein the MAPK-inhibitor is PD98059.

13. The method according to claim 1, wherein the compound is an antisense oligonucleotide.

14. The method according to claim 1, wherein the compound is an antibody that binds a component of the C/EBPβ signaling pathway.

15. The method according to claim 1, wherein the compound is an antibody that binds C/EBPβ.

16. A method of treating a disorder of cell proliferation in a subject in need of such treatment, comprising administering to the subject a compound that disrupts the C/EBPβ signaling pathway.

17. The method according to claim 16, wherein the disorder is cancer.

18. The method according to claim 16, wherein the compound binds a component of the C/EBPβ signaling pathway.

19. The method according to claim 16, wherein the compound binds the C/EBPβ protein.

20. The method according to claim 16, wherein the compound interferes with the expression of the C/EBPβ protein.

21. The method according to claim 16, wherein the compound interferes with post-translational phosphorylation of C/EBPβ.

22. The method according to claim 16, wherein the compound disrupts phosphorylation of C/EBPβ protein at a phosphorylation site selected from the group consisting of Thr 217, Ser 276, and Thr 188.

23. The method according to claim 16, wherein the compound is a MAPK-inhibitor.

24. The method according to claim 23, wherein the MAPK-inhibitor is PD98059.

25. The method according to claim 16, wherein the compound is an antisense oligonucleotide.

26. The method according to claim 16, wherein the compound is an antibody that binds a component of the C/EBPβ signaling pathway.

27. The method according to claim 16, wherein the compound is an antibody that binds C/EBPβ.

28. A method of reducing tumor size in a subject in need of such treatment, comprising administering to the subject a compound that disrupts the C/EBPβ signaling pathway.

29. The method according to claim 28, wherein the tumor is benign.

30. The method according to claim 28, wherein the tumor is malignant.

31. The method according to claim 28, wherein the tumor is a papilloma.

32. The method according to claim 28, wherein the compound binds a component of the C/EBPβ signaling pathway.

33. The method according to claim 28, wherein the compound binds the C/EBPβ protein.

34. The method according to claim 28, wherein the compound interferes with the expression of the C/EBPβ protein.

35. The method according to claim 28, wherein the compound interferes with post-translational phosphorylation of C/EBPβ.

36. The method according to claim 28, wherein the compound disrupts phosphorylation of C/EBPβ protein at a phosphorylation site selected from the group consisting of Thr 217, Ser 276, and Thr 188.

37. The method according to claim 28, wherein the compound is a MAPK-inhibitor.

38. The method according to claim 37, wherein the MAPK-inhibitor is PD98059.

39. The method according to claim 28, wherein the compound is an antisense oligonucleotide.

40. The method according to claim 28, wherein the compound is an antibody that binds a component of the C/EBPβ signaling pathway.

41. The method according to claim 28, wherein the compound is an antibody that binds C/EBPβ.

42. A method of screening for an compound that disrupts the C/EBPβ pathway, comprising determining if a test compound alters the level of expression of a component of the C/EBPβ pathway.

43. The method of claim 42, comprising the steps of:
a) detecting the transcriptional or translational product of a recombinant nucleic acid construct comprising a nucleic acid encoding a component of the C/EBPβ pathway, wherein the nucleic acid encoding a component of the C/EBPβ pathway is operatively linked with a reporter gene, and
b) determining whether the amount of the transcriptional or translational product of said reporter gene differs in the presence and absence of the test compound.

44. The method of claim 41, wherein the component of the C/EBPβ pathway is C/EBPβ.

45. A method of screening for a compound that alters the expression of a component of the C/EBPβ pathway in a cell, comprising:
contacting a cell with test compound, the cell comprising a nucleic acid encoding a component of the C/EBPβ pathway operably linked to a reporter gene; and
incubating the cell under conditions appropriate for expression of the reporter gene and
assessing expression of reporter gene, wherein a change in expression of the reporter gene is indicative of the ability of the compound to alter expression
of the component of the C/EBPβ pathway.

46. The method of claim 45, wherein the component of the C/EBPβ pathway is C/EBPβ.

47. A pharmaceutical formulation for the treatment of a disorder of cell proliferation, comprising a compound that disrupts the C/EBPβ pathway, and a pharmaceutically acceptable carrier.
